(19) United States
(12) Patent Application Publication
(10) Pub. No.: US 2015/0064060 A1
(43) Pub. Date: Mar. 5, 2015

(54) Scented windshield washer fluid

(71) Applicants: Travis McCaughey, Nikiski, AK (US); Randy Alan Pennington, Wasilla, AK (US)

(72) Inventors: Travis McCaughey, Nikiski, AK (US); Randy Alan Pennington, Wasilla, AK (US)

(21) Appl. No.: 14/018,603

(22) Filed: Sep. 5, 2013

(51) Int. Cl. A61L 9/14; B60S 1/50

(52) U.S. Cl. CPC A61L 9/14; B60S 1/50
USPC 422/4; 239/6

(57) ABSTRACT

The invention provides and improved way to provide air freshener, scent, fragrance or perfume to an automobile in a milder and evenly applied manner. The additive or premixed fluid is combined with windshield washer fluid. Where it may be dispensed or applied on the exterior of the windshield. Where by it is pulled from the outside of the car and vented into the automobile, for use as and interior air freshener.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field of automotive. More particularly, the present invention is in the technical field of air fresheners. More particularly, the present invention is in the technical field of exterior automotive air fresheners.

Conventional automobile interior air fresheners, such as fragrance soaked pads, liquid scent containers, pressurized and no pressurized hand held spray cans, gels and solids typically have an overwhelming fragrance, until they have been open for a while. Traditional automobile interior air fresheners are sold in high concentrations which make it difficult to get a more pleasing and mild smell. Additionally, using hand held car fresheners can be a safety hazard if the driver has to reach for the product to manually apply it. In addition these manually applied methods can become burdensome. From the simple fact that more thought and effort is used in apply them.

### SUMMARY OF THE INVENTION

The present invention is scented windshield washer fluid.

### BRIEF DESCRIPTION OF THE DRAWING

Not Applicable

### DETAILED DESCRIPTION OF THE INVENTION

The invention is applied to windshield washer fluid. Having a fragrance added to it by the consumer or as a premixed product, to form a scented mixture. The scented mixer is suitable as an exterior car air freshener when applied to the windshield. The amount of additive or desired premixed fluid that is added to the windshield fluid to compose a sufficient mixture is dependent upon a number of factors, windshield washer fluid composition, volume, and desired concentration of application. In further detail, windshield washer additive is composed of but not limited to Ethyl alcohol Ethanol in a 3-7% composition range. Additionally the scented windshield washer fluid is composed of but not limited to 3-8% aromatic compounds. The scented windshield washer fluid does not hinder or impede or affect the ability of the fluids original intent.

The advantage of the present invention include, without limitation, that it is easy, convenient, safe, and distributes scent, perfume, or fragrance effectively. It is easy to use because it is added once to a large volume of windshield washer fluid, or sold in a large volume pre mixed. Being mixed with a larger volume of fluid allows the strength of scent, perfume or fragrance to be adjusted due to dilution. It can be applied from the interior of most standard automobile via the steering wheel windshield washer fluid spray handle or button; this method keeps the drivers from being distracted. Further the way the product is applied, allows for an even distribution by use of fan settings inside the vehicle.

### EXAMPLE

An individual would add a container of windshield wiper fluid additive, or pre mixed washer fluid, to their windshield washer fluid reservoir. They would then apply the windshield wiper fluid to the windshield of their vehicle from inside, while in motion. As the fluid is applied to the windshield, the scent is pulled into the vehicles fresh air intake system where it is then blown through the interior vents. If the vehicle vent system was not turned on then the scent would be unable to enter the vehicle. Additionally, if the vehicle does not draw air in from below the windshield of the vehicle the scented washer fluid will not be drawn into the car properly, reducing its effectiveness. If the car is equipped with a HEPA or other type filter the effectiveness will be reduced.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

The embodiments of the invention in which an exclusive property or privilege is claimed are defined as follow:

1. The method of freshening or deodorizing the interior of a vehicle with an air freshener or deodorizer, which comprises mixing air freshener or deodorizer into the vehicles windshield wiper reservoir, wherein the active ingredient is air freshener or deodorizer.
